(19) United States
(12) Patent Application Publication
(10) Pub. No.: US 2005/0202068 A1
(43) Pub. Date: Sep. 15, 2005

(54) Disposable nonwoven mitt

(76) Inventors: Erik John Hasenoehrl, Loveland, OH (US); Junko Matsumoto, Mason, OH (US); Daniel Burton Sears, Hamilton, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/067,444

(22) Filed: Feb. 25, 2005

Related U.S. Application Data
(60) Provisional application No. 60/552,859, filed on Mar. 12, 2004.

(51) Int. Cl. A61K 9/70

(52) U.S. Cl. 424/443; 442/123

(57) ABSTRACT

A disposable nonwoven mitt is adapted to be retained on a user's finger(s) during use. The disposable nonwoven mitt can be associated with a personal care composition. The disposable, nonwoven mitts can be useful for cleansing and/or treating the skin, hair or other similar keratin-containing surfaces. These mitts can be used by the consumer by either wetting a substantially dry mitt with water and then rubbing the mitt against the skin, hair or other similar keratin-containing surfaces, or taking a wet, disposable, nonwoven mitt and rubbing against the skin, hair or other similar keratin-containing surfaces or taking a moist, disposable, nonwoven mitt and rubbing against the skin, hair or other similar keratin-containing surfaces.

### CROSS REFERENCE TO RELATED APPLICATION(S)

This application claims the benefit of Provisional Application Ser. No. 60/552,859, filed Mar. 12, 2004.

### FIELD OF THE INVENTION

A disposable nonwoven mitt adapted to be retained on a user's finger(s) during use is provided. The disposable nonwoven mitt can be associated with a personal care composition.

### BACKGROUND OF THE INVENTION

Personal care products, particularly cleansing and conditioning products, have traditionally been marketed in a variety of forms such as bar soaps, creams, lotions, and gels. Typically, these products must satisfy a number of criteria to be acceptable to consumers. These criteria include cleansing effectiveness, skin feel, mildness to skin, hair, and ocular mucosae, and lather volume. Ideal personal cleansers should gently cleanse the skin or hair, cause little or no irritation, and should not leave the skin or hair overly dry after frequent use.

It is highly desirable to deliver cleansing and conditioning benefits from a disposable substrate. Disposable products are convenient because they obviate the need to carry or store cumbersome bottles, bars, jars, tubes, and other forms of clutter associated with cleansing products and other products capable of providing therapeutic or aesthetic benefits. Disposable products are also a more sanitary alternative to the use of a sponge, washcloth, or other cleansing implement intended for extensive reuse, because such implements can develop bacterial growth, unpleasant odors, and other undesirable characteristics related to repeated use.

However, while disposable personal care articles, such as disposable washcloths or disposable mitts, are desirable they have their own problems. Retention on the hand or finger of a user of such disposable mitts, especially during vigorous scrubbing, is one such problem. If the mitt is prone to fall off during use, the user is more concerned with retaining the cleansing mitt on their hand or finger instead of actually using the mitt to wash themselves.

The need remains for disposable products which are easy to use and suitable for use by consumers. Furthermore, the need remains for a disposable mitt which is retained on a user's finger (s) such that the consumer can focus on the task at hand, namely washing, cleaning, and conditioning without having to be concerned with retaining the wash mitt on their finger.

### SUMMARY OF THE INVENTION

The present invention relates to a disposable nonwoven mitt adapted to fit around at least one finger on a user's hand, said mitt comprising a first and second nonwoven sheet members, said first and second nonwoven members being secured to each other along at least a portion of a periphery of said nonwoven sheet members to form an interior volume between said nonwoven sheet members and an opening to receive said finger(s) of said user's hand; and wherein at least one of said nonwoven sheet members is adapted to extend below a web of said user's hand when said mitt is placed on said finger(s) of said user's hand to form a flap portion of said mitt; and wherein said flap portion is optionally grasped by a user's thumb during use.

The present invention further relates to a disposable nonwoven mitt adapted to fit around at least one finger on a user's hand, said mitt comprising: a first and second nonwoven sheet members, said first and second nonwoven members being secured to each other along at least a portion of a periphery of said nonwoven sheet members to form an interior volume between said nonwoven sheet members and an opening to receive said finger(s) of said user's hand; and a personal care composition associated with at least one of said first and second nonwoven sheet members.

The present invention further relates to a disposable nonwoven mitt adapted to fit around at least one finger on a user's hand, said mitt comprising a first and second nonwoven sheet members; wherein at least one of said members is a multi-layer nonwoven sheet member; wherein said first and second nonwoven members being secured to each other along at least a portion of a periphery of said nonwoven sheet members to form an interior volume between said nonwoven sheet members and an opening to receive said finger(s) of said user's hand; and a personal care composition associated with at least one of said first and second nonwoven sheet members.

The present invention further relates methods for cleansing, and treating the skin or hair and similar keratin-containing surfaces of the body, primarily skin and hair using the mitts described herein.

The present invention further relates kits containing a plurality of the disposable nonwoven mitt that has a personal care composition associated with the mitt. In addition, the present invention also covers kits containing a plurality of the disposable nonwoven mitt that has no treatment and, or cleansing composition on it wherein the mitt is packaged with certain types of compositions that during use effectuate cleansing and, or treating the skin, hair, and similar keratin-containing surfaces of the body

### BRIEF DESCRIPTION OF THE DRAWINGS

FIG. 1 is a perspective view of a mitt of the present invention.

FIG. 2 is a perspective view of a mitt of the present invention.

FIG. 3a is a perspective view of a mitt of the present invention.

FIG. 3b is a cross-sectional view of a section of the mitt shown in FIG. 3a.

FIG. 3c is an enlarged view of a portion of the mitt shown in FIG. 3b.

FIG. 4 is a perspective view of a mitt of the present invention.

### DETAILED DESCRIPTION OF THE INVENTION

The instant mitts, and methods of the present invention, are suitable for use by a user, in personal cleansing. Due to the ease and simple method of use a user is able to clean or condition their skin, with the instant invention.

By “composition associated with the mitt or nonwoven sheet member(s)”, as used herein, means compositions that are applied to or inside of the individual fibers prior to forming the mitt, permeated into the mitt, coated onto, within or adjacent to the exposed surfaces of the mitt, within or adjacent to the interior surfaces of the mitt. The composition can be releasably associated with the mitt.

The term “treatment agent or composition”, as used herein, means a formulation to deliver cosmetic and therapeutic benefit to keratin-containing surfaces of the body, primarily skin and hair.

The term “disposable” is used herein in its ordinary sense to mean an article that is disposed or discarded after a limited number of usage events, preferably less than 5, more preferably less than about 3, and even more preferably less than about 2 entire usage events.

The term “fluid” is used herein to mean “fluids” selected from the group consisting of water, mono- and polyhydric alcohols (glycerin, propylene glycol, ethanol, isopropanol, etc.), hydrocarbon oils such as mineral oil, silicone fluids, also triglyceride oils, also fluid resins such as silicone MQ resins, esters and ethers of hydrocarbons, alcohols, perfume, fragrance oils, natural oils such as terpenes, various tree and plant oils, as well as mixtures of the above and can contain other components dissolved or dispersed within them, or in addition to them.

The phrase “substantially free of” as used herein, means that the composition comprises less than about 3%, preferably less than about 1%, more preferably less than about 0.5%, even more preferably less than about 0.25%, and still more preferably less than about 0.1%, even still more preferably less than 0.01% by weight of the composition, of the stated ingredient.

The term “water-activated,” as used herein, means that some of the articles of the present invention are presented to the consumer in a form to be used when wetted with a fluid. It is found that these articles produce a benefit or are “activated” by either contacting them with a fluid or producing them with a fluid and then further subjecting the article to mechanical forces, such as rubbing.

The term “substantially dry,” as used herein, means that prior to use the mitt is substantially free of fluid and generally feels dry to the touch. As used herein, “substantially dry” means that the mitts of the present invention exhibit a Moisture Retention of of less than about 0.95 gms, preferably less than about 0.75 gms, even more preferably less than about 0.5 gms, still more preferably less than about 0.25 gms, still even more preferably less than about 0.15 gms, and even still even more preferably less than about 0.1 gms. The determination of the Moisture Retention is discussed later. Thus, the “substantially dry” mitts of the present invention will generally comprise less than about 20% by weight of the dry mitt of fluid, preferably from 4% to about 20% by weight of the dry mitt of fluid, and more preferably from about 4% to about 16% by weight of the dry mitt of fluid.

The term “moist,” as used herein, means that prior to use the mitt can feel relatively dry to the touch and still contain high fluid content. Thus, the “moist” articles of the present invention will generally comprise from about 20% to about 40% by weight of the dry mitt of fluid.

The term “wet” means that prior to use the mitt can feel wet to the touch and contain high fluid content. The weight percent of fluid in the “wet” article is based on the total weight of the composition. The weight is expressed as a by weight of fluid. Thus, the “wet” mitts of the present invention will generally comprise from about greater than 40% by weight of the dry mitt of fluid, preferably from 40% to about 95% by weight of the dry mitt of fluid, and more preferably from about 50% to about 80% by weight of the dry mitt of fluid.

Disposable Nonwoven Mitt

The disposable nonwoven mitt can be used by individuals primarily for cleansing and, or treatment of skin, hair or other similar keratin-containing surfaces including skin, hair, finger, feet and toe nails. The disposable nonwoven mitts of the present invention comprise nonwoven sheet members. The mitt can be substantially dry, moist or wet. The mitt comprises at least two nonwoven sheet members. The nonwoven sheet members are secured to each other along at least a portion of the periphery of the nonwoven sheet members. The securing of the nonwoven sheet members form an interior volume between the sheet members so that there is an opening to receive at least one finger of the user's hand, preferably at least two fingers, even more preferably at least three fingers, still even more preferably at least four fingers of the user's hand.

In one preferred embodiment of the disposable nonwoven mitt, the nonwoven sheet members are adapted to extend below the web between the fingers of a user's hand to form a flap portion of the mitt. The flap portion can be optionally grasped by the user's thumb during use. Preferably, the nonwoven sheet members of the disposable nonwoven mitt do not extend over the entire hand nor extend over the entire palm of the hand.

In another preferred embodiment, at least one of the nonwoven sheet members of the disposable nonwoven mitt can be a multi-layer nonwoven sheet member. The multi-layer nonwoven sheet members can comprise a batting layer.

In another embodiment the disposable nonwoven mitt has a personal care composition associated with the mitt. Without being limited, the personal care composition can be applied to or inside of the individual fibers prior to forming the mitt, permeated into the mitt, coated onto, within or adjacent to the exposed surfaces of the mitt, within or adjacent to the interior surfaces of the mitt or releasably associated with the mitt.

In another embodiment of the current invention, the disposable nonwoven mitt is ergonomically correct for grasping by the thumb and wherein there is an opening to receive at least about one finger of the user's hand wherein the mitt can be used by individuals primarily for cleansing and, or treatment of skin, hair or other similar keratin-containing surfaces including feet, finger(s) and toe nails. The nonwoven disposable mitt has a curved edge at the top and the bottom of the mitt. The nonwoven disposable mitt comprising a particular ratio of a top curved radius and a bottom curved radius has a superior ability to reach and effectively contact hard to cleanse and treat areas including but not limited to the skin, hair or other similar keratin-containing surfaces including feet, finger(s) and toe nails.

The ratio of the top curved radius and bottom curved radius is preferably from about 1.05 to about 3, more preferably from about 1.25 to about 2.75, even more preferably from about 1.5 to about 2.5, and still even more preferably from about 1.75 to about 2.25. The radius of the top curved and a bottom curved radius is measured using and Autocad software radius measurement tool. In addition, the length to width ratio of the nonwoven disposable mitt also aids in the superior ability to reach and effectively contact hard to cleanse and treat areas. The ratio of length to width is preferably from about 1.4 to about 1, more preferably from about 1.35 to about 1.1. Additionally, the nonwoven disposable mitt has a length that is preferably greater than about 85 mm, more preferably greater than about 90 mm, and even more preferably greater than about 100 mm.

Nonwoven Sheet Members

The nonwoven sheet members tend to enhance cleansing. The nonwoven sheet members can have the same or differing textures such as a soft texture, exfoliating texture or combinations thereof on each side of the sheet members. The nonwoven sheet members may act as an efficient lathering and exfoliating implement. By physically coming into contact with the skin or hair, the nonwoven sheet members significantly aids in cleansing and removal of dirt, makeup, dead skin, and other debris. In preferred personal care embodiments, however, the substrate is non-scouring or nonabrasive to the skin.

The first and second nonwoven sheet members comprise fibers. The nonwoven sheet members may comprise a variety of both natural and synthetic fibers or materials. As used herein, “natural fibers” are those derived from plants, animals, insects or byproducts of plants, animals, and insects. The conventional base starting material is preferably a fibrous web comprising any of the common synthetic or natural textile-length fibers, or combinations thereof. The fiber is selected from the group consisting of monocomponent fibers, multicomponent fibers, multiconstituent fibers, capillary channel fibers, hollow fibers, shaped or lobed fibers and combinations thereof.

Nonlimiting examples of natural materials useful in the present invention include, but are not limited to, silk fibers, keratin fibers and cellulosic fibers. Nonlimiting examples of keratin fibers include those selected from the group consisting of wool fibers, camel hair fibers, and the like. Nonlimiting examples of cellulosic fibers include those selected from the group consisting of wood pulp fibers, cotton fibers, hemp fibers, jute fibers, rayon fibers, flax fibers, and combinations thereof. Cellulosic fiber materials are preferred in the present invention. Cellulosic fiber materials are preferred in the present invention such as paper, rayon and Tencile™. Nonlimiting examples of synthetic materials useful in the present invention include those selected from the group consisting of acetate fibers, acrylic fibers, cellulose ester fibers, modacrylic fibers, polyamide fibers, polyester fibers, polyolefin fibers, polyvinyl alcohol fibers, rayon fibers, Tencel™ fibers, polyethylene foam, polyurethane foam, and combinations thereof. Examples of suitable synthetic materials include acrylics such as Acrilan™, Creslan™, and the acrylonitrile-based fiber, Orlon™; cellulose ester fibers such as cellulose acetate, Arnel™, and Acele™; polyamides such as nylons (e.g., nylon 6, nylon 66, nylon 610, and the like); polyesters such as Fortrel™, Kodel™, and the polyethylene terephthalate fiber, polybutylene terephthalate fiber, Dacron™; polyolefins such as polypropylene, polyethylene; polyvinyl acetate fibers; and combinations thereof. These and other suitable fibers and the nonwovens prepared there from are generally described in Riedel, “Nonwoven Bonding Methods and Materials,” Nonwoven World (1987); The Encyclopedia Americana, vol. 11, pp. 147-153, and vol. 26, pp. 566-581 (1984); U.S. Pat. No. 4,891,227, to Thaman et al., issued Jan. 2, 1990; and U.S. Pat. No. 4,891,228. As used herein, “nonwoven” means that the layer comprises fibers which are not woven into a fabric but rather are formed into a sheet, mat, or pad layer. The fibers can either be random (i.e., randomly aligned) or they can be carded (i.e., combed to be oriented in primarily one direction). Nonwoven sheet members made from synthetic materials useful in the present invention can be obtained from a wide variety of commercial sources.

More preferred synthetic fibers for the nonwoven sheet members are solid staple polyester fibers, which comprise polyethylene terephthalate homopolymers. Suitable synthetic materials may include solid single component and multicomponent synthetic fibers, i.e., more than one type of material making up the fibers. The synthetic fibers may comprise bicomponent or dual component fibers. Such bicomponent fibers may have a core and a sheath configuration or a side-by-side configuration. In either instance, the nonwoven sheet members may comprise either a combination of fibers comprising the above-listed materials or fibers which themselves comprise a combination of the above-listed materials.

For the core-sheath fibers, preferably, the cores comprise materials selected from the group consisting of polyesters, polyolefins having a T_{g} or melting point of at least about 10° C. higher than the sheath material, and combinations thereof. Conversely, the sheaths of the bicomponent fibers preferably comprise materials selected from the group consisting of polyolefins having a T_{g} or melting point of at least about 10° C. lower than the core material, polyesters polyolefins having a T_{g} or melting point of at least about 10° C. lower than the core material, and combinations thereof.

In any instance, side-by side configuration or core-sheath configuration, the fibers of the nonwoven sheet members may exhibit a helical or spiral configuration, particularly the bicomponent type fibers.

A preferred synthetic material for scouring nonwoven sheet members may comprise nylon fibers. A more preferred synthetic material comprises nylon fibers formed into a scrim layer having additional nylon fibers bonded thereto such that the additional fibers form arcs on the scrim layer.

Natural material nonwovens useful in the present invention may be obtained from a wide variety of commercial sources. Nonlimiting examples of suitable commercially available paper layers useful herein include Airtex®, an embossed airlaid cellulosic layer having a base weight of about 71 gsy, available from James River, Green Bay, Wis.; and Walkisoft®, an embossed airlaid cellulosic having a base weight of about 75 gsy, available from Walkisoft U.S.A., Mount Holly, N.C.

Additional suitable nonwoven sheet members include, but are not limited to, those disclosed in U.S. Pat. No. 4,447,294, issued to Osborn on May 8, 1984; U.S. Pat. No. 4,603,176 issued to Bjorkquist on Jul. 29, 1986; U.S. Pat. No. 4,981,557 issued to Bjorkquist on Jan. 1, 1991; U.S. Pat. No. 5,085,736 issued to Bjorkquist on Feb. 4, 1992; U.S. Pat. No. 5,138,002 issued to Bjorkquist on Aug. 8, 1992; U.S. Pat. No. 5,262,007 issued to Phan et al. on Nov. 16, 1993; U.S. Pat. No. 5,264,082, issued to Phan et al. on Nov. 23, 1993; U.S. Pat. No. 4,637,859 issued to Trokhan on Jan. 20, 1987; U.S. Pat. No. 4,529,480, issued to Trokhan on Jul. 16, 1985; U.S. Pat. No. 4,687,153 issued to McNeil on Aug. 18, 1987; U.S. Pat. No. 5,223,096, issued to Phan et al. on Jun. 29, 1993 and U.S. Pat. No. 5,679,222, issued to Rasch et al. on Oct. 21, 1997.

Additional suitable materials include, but are not limited to, formed films and composite materials, e.g., multiply materials containing formed films. Preferably, such formed films comprise plastics which tend to be soft to the skin. Suitable soft plastic formed films include, but are not limited to, polyolefins such as low density polyethylenes (LDPE).

Methods of making nonwovens are well known in the art. Generally, these nonwovens can be made by air-laying, water-laying, meltblowing, coforming, spunbonding, or carding processes in which the fibers or filaments are first cut to desired lengths from long strands, passed into a water or air stream, and then deposited onto a screen through which the fiber-laden air or water is passed. The resulting layer, regardless of its method of production or composition, is then subjected to at least one of several types of bonding operations to anchor the individual fibers together to form a self-sustaining mitt. In the present invention the nonwoven sheet members can be prepared by a variety of processes including, but not limited to, meltblowing, spunbonding, air-entanglement, hydroentanglement, thermal bonding, selective mechanical deformation as described in co-pending U.S. application Ser. No. 10/737,640 filed on Dec. 16, 2003 and combinations of these processes. Preferably, the nonwoven sheet members are prepared by selective mechanical deformation.

The nonwoven sheet members can also be secured, by either chemical or physical means around at least a portion of the periphery of the nonwoven sheet members. Preferably the flap portion of a nonwoven sheet member that is located below the web between the finger (s) of the user is not secured to another nonwoven sheet member. Secured by chemical means can be accomplished by causing the periphery of the nonwoven sheet members to join when they are chemically similar, in which case adhesion may be assisted by heat, pressure, solvent, adhesives or combinations thereof. Heat and pressure can be provided by various processing techniques which are well known. Securing by physical means can be accomplished by needle punching, ultrasonic bonding, high pressure bonding, thermal bonding, crimping, stitching, or combinations thereof. The securing by physical means involves the interpenetration of at least a portion of the periphery one nonwoven sheet member into and around at least a portion of the periphery of a second nonwoven sheet member.

Batting Layer

In an embodiment of the present invention at least one of the nonwoven sheet members can comprise a multi-layered nonwoven sheet member comprising a batting layer. The batting layer preferably comprises synthetic materials. As used herein, “synthetic” means that the materials are obtained primarily from various man-made materials or from natural materials that have been further altered. Suitable synthetic materials include, but are not limited to, acetate fibers, acrylic fibers, cellulose ester fibers, modacrylic fibers, polyamide fibers, polyester fibers, polyolefin fibers, polyvinyl alcohol fibers, rayon fibers, polyethylene foam, polyurethane foam, and combinations thereof. Preferred synthetic materials, particularly fibers, may be selected from the group consisting of nylon fibers, rayon fibers, Tencel™ fibers, polyolefin fibers, polyester fibers, and combinations thereof. Preferred polyolefin fibers are fibers selected from the group consisting of polyethylene, polypropylene, polybutylene, polypentene, and combinations and copolymers thereof. More preferred polyolefin fibers are fibers selected from the group consisting of polyethylene, polypropylene, and combinations and copolymers thereof. Preferred polyester fibers are fibers selected from the group consisting of polyethylene terephthalate, polybutylene terephthalate, polycyclohexylenedimethylene terephthalate, and combinations and copolymers thereof. More preferred polyester fibers are fibers selected from the group consisting of polyethylene terephthalate, polybutylene terephthalate, and combinations and copolymers thereof. Most preferred synthetic fibers comprise solid staple polyester fibers that comprise polyethylene terephthalate homopolymers. Suitable synthetic materials may include solid single component (i.e., chemically homogeneous) fibers, multiconstituent fibers (i.e., more than one type of material making up each fiber), and multicomponent fibers (i.e., synthetic fibers which comprise two or more distinct filament types which are somehow intertwined to produce a larger fiber), and combinations thereof. Preferred fibers include bicomponent fibers, multiconstituent fibers, and combinations thereof. Such bicomponent fibers may have a core-sheath configuration or a side-by-side configuration. In either instance, the batting layer may comprise either a combination of fibers comprising the above-listed materials or fibers which themselves comprise a combination of the above-listed materials.

The batting layer may also comprise natural fibers. Suitable natural fibers are described above in the nonwoven sheet members section.

Furthermore, the fibers of the batting layer preferably have an average thickness of from about 0.5 microns to about 150 microns. More preferably, the average thickness of the fibers are from about 5 microns to about 75 microns. In an even more preferred embodiment, the average thickness of the fibers are from about 8 microns to about 40 microns. Furthermore, the fibers of the batting layer may be of varying sizes, i.e., the fibers of the batting layer may comprise fibers having different average thicknesses. Also, the cross section of the fibers can be round, flat, oval, elliptical or otherwise shaped.

In another embodiment, the batting layer of the present invention may comprise a composite material, i.e., a material having one or more plies of the same or different suitable materials merely superimposed physically, joined together continuously (e.g., laminated, etc.) or in a discontinuous pattern, or by bonding at the external edges (or periphery) of the layer and/or at discrete loci. The term composite means that at least one layer did not exist as a distinct layer apart from the article, said layer being formed during a processing step involving mixing of two or more components or compositions to form a heterogeneous mixture. The term laminate means at least one layer existed apart from the article and is combined to form an article by a lamination process, including thermal (including ultrasonic) and adhesive bonding lamination processes. For example, the batting layer may further comprise composite materials selected from the group consisting of fibrous nonwovens, sponges, foams, reticulated foams, polymeric nets, scrims, vacuum-formed laminates, formed films and formed film composite materials. It is preferred that the batting layer comprises a formed film composite material comprising at least one formed film and at least one nonwoven wherein the layer is vacuum formed. A suitable formed film composite material includes, but is not limited to a vacuum laminated composite formed film material formed by combining a carded polypropylene nonwoven having a basis weight of 30 gsm with a formed film.

Without being limited by theory, the batting layer is especially useful as a skin contact side in the mitt of the present invention due to its mild exfoliating properties. Furthermore, the preferred synthetic fibers which make up the batting layer are largely hydrophobic thus allowing water to readily flow through the mitt.

Additional Nonwoven Sheet Members

In another embodiment, the mitt of the present invention may comprise one or more additional nonwoven sheet members which one having ordinary skill in the art would recognize as separate and distinct from the first and second nonwoven sheet members yet which are attached to these nonwoven sheet members at some point. The additional nonwoven sheet members are suitable for enhancing the overall cleansing and treatment of the surface to be cleansed and/or therapeutically treated. Also, the additional nonwoven sheet members are suitable for enhancing the soft feel of the either side of the disposable nonwoven mitt which contacts the surface to be cleansed and/or therapeutically treated. In any instance, these additional nonwoven sheet members may also be referred to as consecutively numbered nonwoven sheet members in addition to the two essential nonwoven sheet members of the mitts of the present invention, e.g., third nonwoven sheet member, fourth nonwoven sheet member, etc. Suitable additional nonwoven sheet members may comprise those materials which are disclosed above as suitable for the first and second nonwoven sheet members.

Personal Care Compositions

The present invention is for a disposable nonwoven mitt used by individuals preferably for cleansing and or treatment of skin, hair or other and similar keratin-containing surfaces including skin, hair and finger and toe nails. These disposable nonwoven mitts can encompass the use of personal care compositions that are associated with the nonwoven sheet members of the present invention. The personal care compositions of the present invention are selected from the group consisting of lathering cleansing agents, non-lathering cleansing agents, skin treatment agents, skin conditioning agents, and mixtures thereof. Non-limiting examples of personal care compositions which are suitable for use in the compositions of the present invention are disclosed in U.S. application Ser. No. 60/516,502, filed on Oct. 31, 2003. The present invention can comprise mitts that combine two or more different personal care compositions into a single mitt.

A. Lathering Cleansing Agents

The compositions of the present invention also can comprise one or more lathering cleansing agents that are associated with at least one of the nonwoven sheet members of the present invention. Thus the lathering cleansing agents can be associated with the nonwoven sheet member. Generally this will be done prior to the point of use of the mitt, i.e., the lathering cleansing agents will be combined with the mitt before the mitt is ultimately wetted for use. Preferred articles of the present invention are either wet, moist, or substantially dry. The lathering cleansing agents can be used on skin, hair and similar keratinous regions.

The lathering cleansing agents create a lather which is used to wash a small portion of the skin generally using lower amounts of fluid. Lathering cleansing agents are particularly useful for facial cleansing at a sink. By a lathering cleansing agent is meant a lathering surfactant, which when combined with a fluid and mechanically agitated generates a foam or lather sufficient to cause the article, as a whole, to lather. Preferably, these surfactants or combinations of surfactants should be mild, which means that these surfactants provide sufficient cleansing or detersive benefits but do not overly dry the skin or hair, and yet meet the lathering criteria described above. Generally the lathering cleansing agents will preferably comprise no more than about 250 weight percent by weight of the mitt of a lathering surfactant. Preferably, the agents of the present invention comprises no more than about 100 weight percent, more preferably no more than about 75 weight percent, and still more preferably no more than about 50 weight percent by weight of the mitt of a lathering surfactant. Generally the lathering cleansing agents will preferably comprise at least 0.5 weight percent by weight of the mitt of a lathering surfactant. Preferably, the agents of the present invention comprises at least 0.6 weight percent, more preferably at least 0.75 weight percent, and still more preferably at least 1 weight percent by weight of the mitt of a lathering surfactant.

A wide variety of lathering surfactants are useful for the lathering cleansing agents described herein and include those selected from the group consisting of anionic lathering surfactants, nonionic lather surfactants, amphoteric lathering surfactants, and mixtures thereof. Non-limiting examples of lathering surfactants useful in the compositions of the present invention are disclosed in U.S. Pat. No. 6,280,757, to McAtee et al., issued Aug. 28, 2001. Generally, the lathering surfactants do not strongly interfere with deposition of any conditioning agents that are present, e.g., are fairly water soluble, and usually have an HLB value of above 10. Cationic surfactants can also be used as optional components, provided they do not negatively impact the overall lathering characteristics of the required lathering surfactants.

Suitable Surfactants for the lathering cleansing agents described above include the following surfactants:

Anionic Lathering Surfactants

Non-limiting examples of anionic lathering surfactants useful in the compositions of the present invention are disclosed in McCutcheon's, Detergents and Emulsifiers, North American edition (1986), published by allured Publishing Corporation; McCutcheon's, Functional Materials, North American Edition (1992); and U.S. Pat. No. 3,929,678, to Laughlin et al., issued Dec. 30, 1975.

A wide variety of anionic lathering surfactants are useful herein. Non-limiting examples of anionic lathering surfactants include those selected from the group consisting of sarcosinates, sulfates, isethionates, taurates, phosphates, lactylates, glutamates, and mixtures thereof. Amongst the isethionates, the alkoyl isethionates are preferred, and amongst the sulfates, the alkyl and alkyl ether sulfates are preferred.

Other anionic materials useful herein include are fatty acid soaps (i.e., alkali metal salts, e.g., sodium or potassium salts) typically having from a fatty acid having about 8 to about 24 carbon atoms, preferably from about 10 to about 20 carbon atoms. These fatty acids used in making the soaps can be obtained from natural sources such as, for instance, plant or animal-derived glycerides (e.g., palm oil, coconut oil, soybean oil, castor oil, tallow, lard, etc.) The fatty acids can also be synthetically prepared. Soaps and their preparation are described in detail in U.S. Pat. No. 4,557,853.

Other anionic materials include phosphates such as monoalkyl, dialkyl, and trialkylphosphate salts. Non-limiting examples of preferred anionic lathering surfactants useful herein include those selected from the group consisting of sodium lauryl sulfate, ammonium lauryl sulfate, ammonium laureth sulfate, sodium laureth sulfate, sodium trideceth sulfate, ammonium cetyl sulfate, sodium cetyl sulfate, ammonium cocoyl isethionate, sodium lauroyl isethionate, sodium lauroyl lactylate, triethanolamine lauroyl lactylate, sodium caproyl lactylate, sodium lauroyl sarcosinate, sodium myristoyl sarcosinate, sodium cocoyl sarcosinate, sodium lauroyl methyl taurate, sodium cocoyl methyl taurate, sodium lauroyl glutamate, sodium myristoyl glutamate, and sodium cocoyl glutamate and mixtures thereof.

Especially preferred for use herein is ammonium lauryl sulfate, ammonium laureth sulfate, sodium lauroyl sarcosinate, sodium cocoyl sarcosinate, sodium myristoyl sarcosinate, sodium lauroyl lactylate, and triethanolamine lauroyl lactylate.

Non-Ionic Lathering Surfactants

Non-limiting examples of nonionic lathering surfactants for use in the compositions of the present invention are disclosed in McCutcheon's, Detergents and Emulsifiers, North American edition (1986), published by allured Publishing Corporation; and McCutcheon's, Functional Materials, North American Edition (1992).

Nonionic lathering surfactants useful herein include those selected from the group consisting of alkyl glucosides, alkyl polyglucosides, polyhydroxy fatty acid amides, alkoxylated fatty acid esters, lathering sucrose esters, amine oxides, and mixtures thereof.

Non-limiting examples of preferred nonionic surfactants for use herein are those selected form the group consisting of C₈-C₁₄ glucose amides, C₈-C₁₄ alkyl polyglucosides, sucrose cocoate, sucrose laurate, lauramine oxide, cocoamine oxide and mixtures thereof.

Amphoteric Lathering Surfactants

The term “amphoteric lathering surfactant,” as used herein, is also intended to encompass zwitterionic surfactants, which are well known to formulators skilled in the art as a subset of amphoteric surfactants.

A wide variety of amphoteric lathering surfactants can be used in the compositions of the present invention. Particularly useful are those which are broadly described as derivatives of aliphatic secondary and tertiary amines, preferably wherein the nitrogen is in a cationic state, in which the aliphatic radicals can be straight or branched chain and wherein one of the radicals contains an ionizable water solubilizing group, e.g., carboxy, sulfonate, sulfate, phosphate, or phosphonate.

Non-limiting examples of amphoteric surfactants useful in the compositions of the present invention are disclosed in McCutcheon's, Detergents and Emulsifiers, North American edition (1986), published by allured Publishing Corporation; and McCutcheon's, Functional Materials, North American Edition (1992).

Non-limiting examples zwitterionic surfactants are those selected from the group consisting of betaines, sultaines, hydroxysultaines, alkyliminoacetates, iminodialkanoates, aminoalkanoates, and mixtures thereof.

Preferred lathering surfactants for use herein are the following, wherein the anionic lathering surfactant is selected from the group consisting of ammonium lauroyl sarcosinate, sodium trideceth sulfate, sodium lauroyl sarcosinate, ammonium laureth sulfate, sodium laureth sulfate, ammonium lauryl sulfate, sodium lauryl sulfate, ammonium cocoyl isethionate, sodium cocoyl isethionate, sodium lauroyl isethionate, sodium cetyl sulfate, sodium lauroyl lactylate, triethanolamine lauroyl lactylate, and mixtures thereof, wherein the non-ionic lathering surfactant is selected from the group consisting of lauramine oxide, cocoamine oxide, decyl polyglucose, lauryl polyglucose, sucrose cocoate, C₁₂₋₁₄ glucosamides, sucrose laurate, and mixtures thereof; and wherein the amphoteric lathering surfactant is selected from the group consisting of disodium lauroamphodiacetate, sodium lauroamphoacetate, cetyl dimethyl betaine, cocoamidopropyl betaine, cocoamidopropyl hydroxy sultaine, and mixtures thereof.

B. Non-Lathering Cleansing Agents

The compositions of the present invention also can comprise one or more non-lathering cleansing agents that are associated with at least one of the nonwoven sheet members. Preferred articles of the present invention are either wet, moist, or substantially dry and the non-lathering cleansing agents comprise a sufficient amount of one or more non-lathering surfactants such that the agents do not generate a substantial lather.

Non-lathering mitts can be used for exfoliation, wiping mitts (e.g., wet mitts, refreshment mitts) where lather is not desirable and to lie on skin.

Nonlimiting examples of these non-lathering surfactants are: polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, PEG-100 stearate, polyoxyethylene 20 sorbitan trioleate (Polysorbate 85), sorbitan monolaurate, polyoxyethylene 4 lauryl ether sodium stearate, polyglyceryl-4 isostearate, hexyl laurate, PPG-2 methyl glucose ether distearate, ceteth-10, diethanolamine cetyl phosphate, glyceryl stearate, PEG-100 stearate, and mixtures thereof.

C. Skin Conditioning Agents

The compositions of the present invention can comprise a skin conditioning agent that is useful for providing a conditioning benefit to the skin, hair and other parts of the body with keratin-containing tissue. These mitts are generally considered non-lathering and such mitts can be wet, moist or dry as previously defined. The non-lathering cleansing agents are preferably used on skin, hair and similar keratinous regions.

The skin conditioning agent can comprise no more than about 1600 weight percent, preferably no more than about 1000 weight percent, more preferably no more than about 800 weight percent, and most preferably no more than about 600 weight percent by weight of the mitt of a skin conditioning agent. The skin conditioning agent can comprise at least 0.05 weight percent, preferably at least 15 weight percent, more preferably at least 15 weight percent, and most preferably no more than about 60 weight percent by weight of the mitt of a skin conditioning agent.

The skin conditioning agent useful in the present invention can comprise: a water soluble conditioning agent; an oil soluble conditioning agent; a conditioning emulsion; or any combination or permutation of the three. The oil soluble conditioning agent is selected from one or more oil soluble conditioning agents such that the weighted arithmetic mean solubility parameter of the oil soluble conditioning agent is less than or equal to 10.5. The water soluble conditioning agent is selected from one or more water soluble conditioning agents such that the weighted arithmetic mean solubility parameter of the water soluble conditioning agent is greater than 10.5. It is recognized, based on this mathematical definition of solubility parameters, that it is possible, for example, to achieve the required weighted arithmetic mean solubility parameter, i.e. less than or equal to 10.5, for an oil soluble conditioning agent comprising two or more compounds if one of the compounds has an individual solubility parameter greater than 10.5. Conversely, it is possible to achieve the appropriate weighted arithmetic mean solubility parameter, i.e. greater than 10.5, for a water soluble conditioning agent comprising two or more compounds if one of the compounds has an individual solubility parameter less than or equal to 10.5.

Solubility parameters are well known to the formulation chemist of ordinary skill in the art and are routinely used as a guide for determining compatibilities and solubilities of materials in the formulation process. See “Solubility Effects in Product, Package, Penetration, and Preservation”, Cosmetics and Toiletries vol. 103, p 47-69, (October 1988).

Non-limiting examples of useful conditioning agents include those selected from the group consisting of petrolatum, fatty acids, esters of fatty acids, fatty alcohols, ethoxylated alcohols, polyol polyesters, glycerine, glycerin mono-esters, glycerin polyesters, epidermal and sebaceous hydrocarbons, lanolin, straight and branched hydrocarbons, silicone oil, silicone gum, vegetable oil, vegetable oil adduct, hydrogenated vegetable oils, nonionic polymers, natural waxes, petrolatum, petrolatum derivatives, synthetic waxes, polyolefinic glycols, polyolefinic monoester, polyolefinic polyesters, cholesterols, cholesterol esters, triglycerides and mixtures thereof.

More particularly, the conditioning agent may be selected from the group consisting of paraffin, mineral oil, petrolatum, stearyl alcohol, cetyl alchohol, cetearyl alcohol, behenyl alcohol, C10-30 polyesters of sucrose, stearic acid, palmitic acid, behenic acid, oleic acid, linoleic acid, myristic acid, lauric acid, ricinoleic acid, steareth-1-100, cetereath 1-100, cholesterols, cholesterol esters, glyceryl tribehenate, glyceryl dipalmitate, glyceryl monostearate, trihydroxystearin, ozokerite wax, jojoba wax, lanolin wax, ethylene glycol distearate, candelilla wax, carnauba wax, beeswax, and silicone waxes.

Mineral oil, which is also known as petrolatum liquid, is a mixture of liquid hydrocarbons obtained from petroleum. See The Merck Index, Tenth Edition, Entry 7048, p. 1033 (1983) and International Cosmetic Ingredient Dictionary, Fifth Edition, vol. 1, p. 415-417 (1993).

Petrolatum, which is also known as petroleum jelly, is a colloidal system comprising nonstraight-chain solid hydrocarbons and high-boiling liquid hydrocarbons. See The Merck Index, Tenth Edition, Entry 7047, p. 1033 (1983); Schindler, Drug. Cosmet. Ind., p. 89, 36-37, 76, 78-80, 82 (1961); and International Cosmetic Ingredient Dictionary, Fifth Edition, Vol. 1, p. 537 (1993).

Nonvolatile silicones such as polydialkylsiloxanes, polydiarylsiloxanes, and polyalkarylsiloxanes are also useful skin conditioning agents. These silicones are disclosed in U.S. Pat. No. 5,069,897, to Orr, issued Dec. 3, 1991.

The skin conditioning agent preferably used in the present invention may also comprise a conditioning emulsion that is useful for providing a conditioning benefit to the skin, hair and similar keratin-containing surfaces during the use of the mitt. The term “conditioning emulsion” as used herein can either mean the combination of an internal phase comprising a water soluble conditioning agent that is enveloped by an external phase comprising an oil soluble agent or the term “conditioning emulsion” as used herein means the combination of an internal phase comprising an oil soluble agent that is enveloped by an external phase comprising a water soluble agent. In preferred embodiments, the conditioning emulsion would further comprise an emulsifier. The conditioning emulsion comprises from about 15% to about 1600%, preferably from about 25% to about 1000%, more preferably from about 50% to about 800%, and most preferably from about 60% to about 600% by weight of said mitt. In a preferred embodiment the conditioning emulsion comprises (i) an internal phase comprising water soluble conditioning agents as described above, and (ii) an external phase comprising oil soluble agents as described hereinbefore in the oil soluble conditioning agent section or hereinafter in the “Materials Used to Increase Lipid Hardness Value” section. In further embodiments, the conditioning emulsion further comprises an emulsifier capable of forming an emulsion of said internal and external phases. Although an emulsifier capable of forming an emulsion of the internal and external phases is preferred in the present invention, it is recognized in the art of skin care formulations that a water soluble conditioning agent can be enveloped by an oil soluble agent without an emulsifier.

D. Skin Treatment Agents

The compositions of the present invention can comprise a skin treatment agent that is useful for providing a therapeutic benefit and/or cosmetic benefit to the skin, hair and similar keratin-containing surfaces during the use of the mitt. These mitts are generally considered non-lathering and such mitts can be wet, moist or substantially dry as previously defined. The skin treatment agents are suitable for application to keratin-containing tissue, that is, they are suitable for use in contact with human keratin-containing tissue without undue toxicity, incompatibility, instability, allergic response, and the like within the scope of sound medical judgment.

The CTFA Cosmetic Ingredient Handbook, Second Edition (1992) describes a wide variety of nonlimiting cosmetic and pharmaceutical ingredients commonly used in the personal care industry, which are suitable for use in the compositions of the present invention. The skin treatment agent can comprise no more than about 1600 weight percent, preferably no more than about 1000 weight percent, more preferably no more than about 800 weight percent, and most preferably no more than about 600 weight percent by weight of the mitt of a skin treatment agent. The skin treatment agent can comprise at least 0.05 weight percent, preferably at least 15 weight percent, more preferably at least 15 weight percent, and most preferably no more than about 60 weight percent by the weight of the mitt of a skin treatment agent.

The skin treatment agents useful in the present invention can comprise compositions comprising the following nonlimiting examples, vitamins, zeolites, peptides, sunscreen actives, terpene alcohols, desquamation actives including a combination of sulfhydryl compounds and zwitterionic surfactants, and a combination of salicylic acid and zwitterionic surfactants, anti-acne actives, anti-\wrinkle actives/anti-atrophy actives, anti-oxidants/radical scavengers, flavonoids, anti-inflammatory agent, anti-cellulite agents, topical anesthetics, tanning actives, chelators, skin lightening agents, antimicrobial and antifungal actives, skin soothing and skin healing actives and mixtures thereof.

Additional nonlimiting examples skin treatment agents include skin firming agents that are materials that produce an immediate firming or tightening sensation shortly after application to the skin. Further, through this tightening or skin tensing effect, these materials may also provide a visible skin smoothing effect, reducing the appearance of skin texture, fine lines, and wrinkles.

Non-limiting examples of skin firming agents useful in the present invention include a variety of natural and synthetic polymers, such as those described in U.S. Pat. No. 6,284,233. For example, protein extracts from wheat and peas, or egg albumin are effective skin firming agents. Other materials such as the silicates described in EP 1008340 are also suitable for use in the compositions of the present invention.

Combination Cleansing and Treatment Compositions

These mitts can comprise at least two separate personal care compositions on a single mitt. The compositions can be added separately or the compositions can be mixed together prior to being associated with the mitt of the present invention.

Additional Ingredients

The compositions of the present invention can comprise a wide range of other optional components. These additional components should be pharmaceutically acceptable. The CTFA Cosmetic Ingredient Handbook, Second Edition, 1992, describes a wide variety of nonlimiting cosmetic and pharmaceutical ingredients commonly used in the skin care industry, which are suitable for use in the compositions of the present invention. Nonlimiting examples of functional classes of ingredients are described at page 537 of this reference. Examples of these and other functional classes include: abrasives, absorbents, anticaking agents, antioxidants, binders, biological additives, buffering agents, bulking agents, chemical additives, colorants, cosmetic astringents, cosmetic biocides, denaturants, drug astringents, external analgesics, film formers, fragrance components, humectants, opacifying agents, pH adjusters, preservatives, propellants, reducing agents, and skin bleaching agents.

Also useful herein are aesthetic components such as fragrances, pigments, colorings, essential oils, skin sensates, astringents, skin soothing agents, and skin healing agents.

In one embodiment of the present invention the personal care composition can comprise xanthan gum. Preferably, the personal care composition is substantially free of xanthan gum. When the personal care composition comprises xanthan gum, the composition when associated with the mitt can be sticky and undesirable.

The compositions used in the present invention may also contain a “fluid” such as water, mono- and polyhydric alcohols (glycerin, propylene-glycol, ethanol, isopropanol, etc.), hydrocarbon oils such as mineral oil, silicone oils having a viscosity, and can contain other components dissolved or dispersed within them, or in addition to them.

Nonlimiting Embodiments of Disposable Nonwoven Mitt

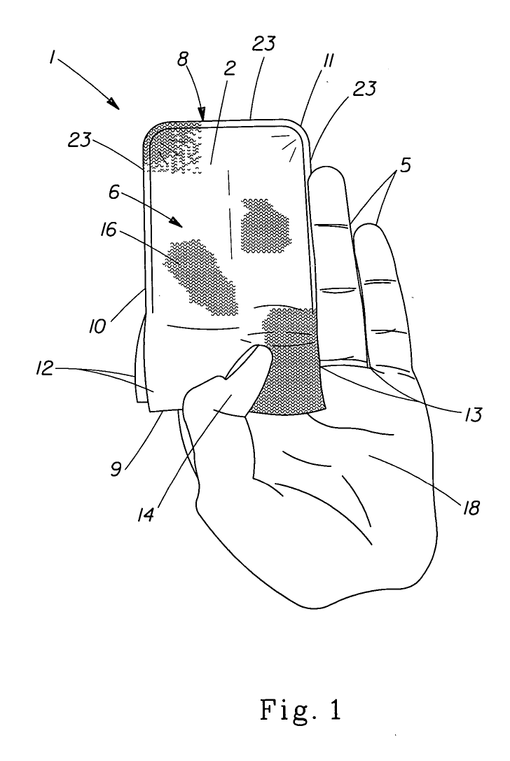

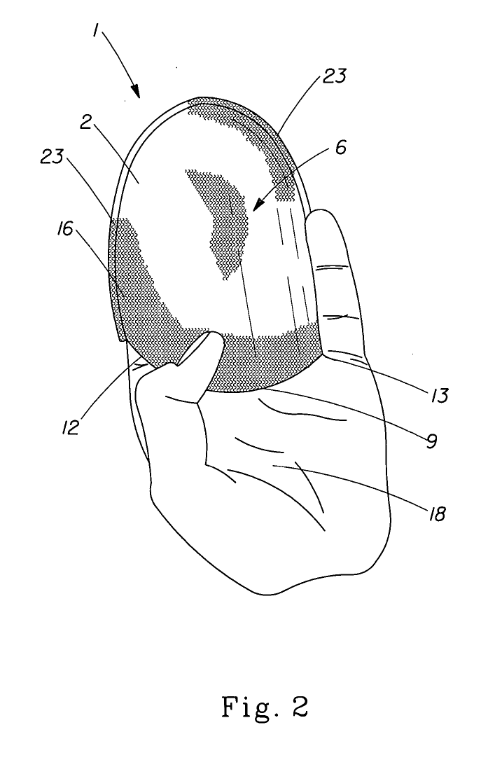

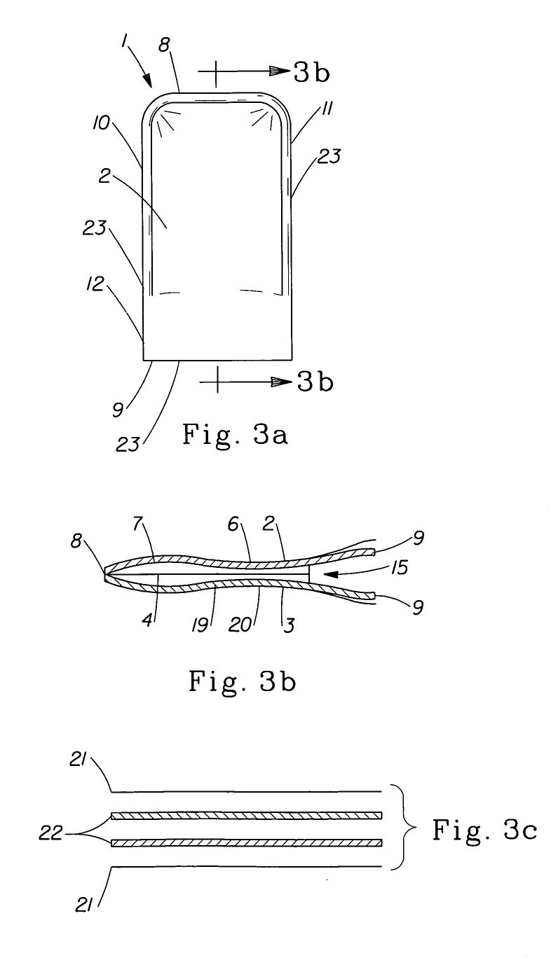

Referring to FIGS. 1 and 3b, there is illustrated one possible embodiment of a disposable nonwoven mitt 1, in accordance with the present invention. The mitt 1 comprises a first nonwoven sheet member 2, which has an exterior surface 6, an interior surface 7, a periphery 23 comprising a top edge 8, a bottom edge 9, a first side edge 10 and a second side edge 11. The first nonwoven sheet member 2, together with the complementary second nonwoven sheet member 3, define an interior volume 4, which is accessed by the user's at least one finger 5 via an opening 15, and wherein at least one of the nonwoven sheet members comprises a flap 12; wherein the flap extends below the web 13 located between the user's fingers 5 wherein the flap 12, which can be located on both the outer side 17 of the hand and the inner side 18 of the hand, is optionally grasped by the user's thumb 14 during use to aid in securing the mitt during use. Preferably at least about one finger, preferably at least about two fingers, more preferably at least about three fingers, even more preferably at least about four fingers, accesses the opening. In another preferred embodiment, referring to FIG. 2, the flap 12 can be located only on the inner side 18 of the hand which is optionally grasped by the user's thumb 14 during use.

The nonwoven disposable mitt 1 in FIG. 1 and FIG. 2 can also comprise a personal care composition 16. In one embodiment of the present invention the personal care composition 16 may be present on a part of the first nonwoven sheet member 2, such as, but not limited to, the exterior surface 6 in the form of stripes, spots, geometric patterns, non-geometric patterns or in a random distribution. In an alternative embodiment, the personal care composition 16 may be present on the entire exterior surface 6 of the first nonwoven sheet member 2. In another alternative embodiment the personal care composition 16 may be present in the interior 7 of the first nonwoven sheet member, and/or the exterior surface 6 of the nonwoven sheet member.

It is to be understood that while in FIG. 1 and FIG. 2 the exterior surface 6 of the first nonwoven sheet member 2 is associated with the personal care composition 16 in other embodiments of the present invention the second 3 nonwoven sheet member may be associated with the personal care composition 16. There is no restriction as to which of the first nonwoven sheet member 2 and the second nonwoven sheet member 3 is associated with the personal care composition 16. It is even possible that both the first 2 and second 3 nonwoven sheet members be associated with the personal care composition 16. Furthermore, the personal care composition 16 may be carried on the exterior surface 6 and 19, interior surface 7 and 20, and/or interior surface 7 and 20 of any nonwoven member as long as a nonwoven member is associated with the personal care composition 16.

Referring to FIG. 3 there is illustrated one possible embodiment, FIG. 3a., of a disposable nonwoven mitt 1, in accordance with the present invention. The mitt 1 comprises a first nonwoven sheet member 2, which has an exterior surface 6, a periphery 23 comprising a top edge 8, a bottom edge 9, a first side edge 10 and a second side edge 11. The mitt 1 in FIG. 3a comprises a bottom edge 9 of the nonwoven sheet members which comprises a flap 12 wherein the flap 12 is not bonded physically or chemically together.

FIG. 3b represents a side view of the mitt 1 shown in FIG. 3a. The mitt 1 comprises a first nonwoven sheet member 2, which has a periphery 23 comprising a top edge 8 and a bottom edge 9, an interior surface 7 of the nonwoven sheet member 2, and an exterior surface 6 of the nonwoven sheet member 2. The mitt 1 also comprises a second nonwoven sheet member 3, which has a periphery 23 comprising a top edge 8 and a bottom edge 9, an interior surface 20 of the nonwoven sheet member 3, and an exterior surface 19 of the nonwoven sheet member 3. The first nonwoven sheet member 2, together with the complementary second nonwoven sheet member 3, define an interior volume 4, which is accessed by the user's at least one finger via an opening 15. Nonwoven sheet member 2 and nonwoven sheet member 3 individually can be single or multi-layered.

In a preferred embodiment shown in FIG. 3c, nonwoven sheet member 2 and nonwoven sheet member 3 comprise a multi-layered embodiment which comprises a nonwoven layer 21 and a batting layer 22. In one embodiment of the present invention the nonwoven layer 21 or the batting layer 22 may be present as the exterior surface 6 of the nowoven sheet member 2. In another an alternative embodiment the nonwoven layer 21 or the batting layer 22 may be present in the interior 7 of the first nonwoven sheet member, and/or the exterior surface 6 of the nonwoven sheet member 2.

It is to be understood that in other embodiments of the present invention the second 3 nonwoven sheet member may have the nonwoven layer 21 or the batting layer 22 present as the exterior surface 19 of the nonwoven sheet member 3. In another alternative embodiment the nonwoven layer 21 or the batting layer 22 may be present in the interior surface 20 of the second nonwoven sheet member, and/or the exterior surface 19 of the nonwoven sheet member 3.

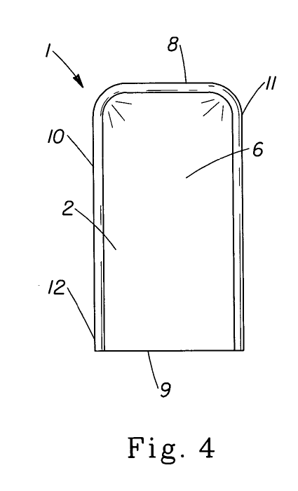

Referring to FIG. 4 there is illustrated one possible embodiment of a disposable nonwoven mitt 1, in accordance with the present invention. The mitt 1 comprises a first nonwoven sheet member 2, which has an exterior surface 6, a periphery 23 comprising a top edge 8, a bottom edge 9, a first side edge 10 and a second side edge 11. The mitt 1 comprises a bottom edge 9 of the nonwoven sheet members which comprises a flap 12 wherein the flap 12 is bonded physically or chemically together.

Moisture Retention Methodology

As described above, the mitts of the present invention can be “substantially dry”. As used herein, “substantially dry” means that the mitts of the present invention exhibit a Moisture Retention of less than about 0.95 gms, preferably less than about 0.75 gms, even more preferably, less than about 0.5 gms, even more preferably less than about 0.25 gms, even still more preferably less than about 0.15 gms, and most preferably, less than about 0.1 gms. The Moisture Retention is indicative of the dry feel that users perceive upon touching the mitts of the present invention as opposed to the feel of “wet” wipes.

In order to determine the Moisture Retention of the present mitts and other disposable nonwoven mitt products, the following equipment and materials are needed.

| | |
|---|---|
| Bounty White Paper Towel | Procter & Gamble SKU 37000 63037 |
| | Basis Weight = 42.14 gsm |
| Balance | Accurate to 0.0 g |
| Lexan | 0.5″ thickness |
| | large enough to cover samples |
| | completely and weighs 1000 g |
| Weight | A 2000 g weight or combination to |
| | equal 2000 g |

Next, weigh two paper towels separately and record each weight. Place one paper towel on flat surface (e.g., lab bench). Place the sample article on top of that towel. Place the other paper towel on top of sample article. Next, place the Lexan and then the 2000 g weight(s) on top of the sandwiched sample article. Wait 1 minute. After the minute, remove weight(s) and Lexan. Weigh the top and bottom paper towel and record the weight.

Calculate the Moisture Retention by subtracting the initial paper towel weight from the final weight (after 1 minute) for both the top and bottom paper towels. Add the weight differences obtained for the top and bottom paper towels. Assuming multiple articles are tested, average the total weight differences to obtain the Moisture Retention.

As described above, the mitts of the present invention can be “substantially dry”, “moist”, or “wet” prior to use. The mitt can feel dry to the touch and still contain high water content. The Moisture Retention is indicative of the dry feel that users perceive upon touching the mitts of the present invention as opposed to the feel of “wet” articles. Thus, mitts of the present invention that feel dry to the touch can have a dry feel relatively independent of the amount of fluid they contain. Articles of the present invention which have a dry feel will exhibit a Moisture Retention of less than about 0.95 gms, preferably less than about 0.75 gms, even more preferably, less than about 0.5 gms, even more preferably less than about 0.25 gms, even still more preferably less than about 0.15 gms, and most preferably, less than about 0.1 gms.

As described above, the mitts of the present invention can be “wet” prior to use. The mitt can feel wet to the touch and contain high fliud content. The weight percent of fliud in the “wet” article is based on the dry weight of the mitt. Thus, the “wet” mitts of the present invention will generally comprise from about greater than 40% by weight of fluid, preferably from 40% to about 95% by weight of the dry mitt of fluid, and more preferably from about 50% to about 80% by weight of the dry mitt of fluid.

The mitt can feel relatively dry to the touch and still contain high fluid content. Thus, the “moist” articles of the present invention will generally comprise from about 20% to about 40% by weight of the dry mitt of fluid.

Multiple Article Embodiment

The present invention also covers kits comprising a plurality of the disposable nonwoven mitt that has a personal care composition associated with the mitt. In addition, the present invention also covers kits comprising a plurality of the disposable nonwoven mitt that has no treatment and, or cleansing composition on it wherein the mitt is packaged with certain types of personal care compositions that during use effectuate cleansing and, or treating the skin, hair, and similar keratin-containing surfaces of the body.

Methods of Manufacture

The mitts of the present invention can be manufactured by separately or simultaneously adding onto the surface of said mitt and, onto or impregnating into said mitt, before or after it has undergone selective mechanical deformation, a cleansing and, or a treatment composition. By “separately” is meant that the cleansing and treatment compositions can be added sequentially, in any order without first being combined together. By “simultaneously” is meant that the cleansing and treatment compositions can be added with or without first being combined together.

The cleansing and/or the treatment composition can be embedded onto or impregnated into the disposable nonwoven mitt using any means known to those skilled in the art. These components can be applied using various spraying, soaking, coating or dipping techniques. Excess surfactant and/or conditioning component can be removed (e.g., by a nipping process). The resulting mitt can remain as wet or can be further processed to be moist or dry using conventional methods known in the art.

Methods of Using Articles

The present invention also relates to a method of cleansing and, or treating the skin, hair or other keratin-containing tissues of the body with a disposable nonwoven mitt of the present invention that is preferably soft due to the structure of the mitt. These methods comprise the steps of wetting with fluid a disposable nonwoven mitt, wiping the article across the area to be cleansed and, or treated with the cleansing and, or a treatment composition. The mitts of the present invention can be wetted with fluid prior to use, already contain fluid prior to use, or not require any fluid in order to use the mitt. The substantially dry mitt is wetted by immersion in fluid or by placing it under a stream of fluid. Lather is generated from the mitt by mechanically agitating and/or deforming the mitt either prior to or during contact of the mitt with the skin, hair or other keratin-containing surfaces.

The resulting lather is useful for cleansing and treating the skin, hair or other keratin-containing surfaces. During the cleansing/treatment process and subsequent optional rinsing with water, the treatment agents and active ingredients are contacted with the skin, hair or other keratin-containing surfaces. Deposition of personal care compositions are enhanced by the physical contact of the disposable nonwoven mitt with the skin, hair or other keratin-containing tissues of the body.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. All exemplified amounts are concentrations by weight of the total personal care compositions, unless otherwise specified.

### Example 1

Lathering Substantially Dry Disposable Nonwoven Mitt

A mitt can be constructed by combining four nonwoven sheet members together. The first nonwoven sheet member can be comprised of bicomponent polyethylene and polypropylene fiber. The second and third nonwoven sheet members can be comprised of a batting layer and the fourth nonwoven sheet member can be comprised of another bicomponent material. The top layer can be coated with an aqueous lathering composition and dried and then add a treatment composition. The bicomponent materials are processed through a selective mechanical deformation process prior to combination of the four nonwoven sheet members. The four nonwoven sheet members are then bonded together using an ultrasonic bonder in a shape described in the Figures above and then rotary cut into individual mitts.

This mitt can be associated with the following personal care composition:

| | | Percent | | |
|---|---|---|---|---|
| | | Composition | | |
| CTFA NAME | | | Chem. | |
| * | CAS NUMBER | | Content | g/member |
| Petrolatum | 8009-03-8 | | 43.76 | 0.2500 |
| Glycerin | 56-81-5 | | 15.91 | 0.0909 |
| Cocamidopropyl | 61789-40-0; | | 10.50 | 0.0600 |
| Betaine | 83138-08-3 | | | |
| Decyl Glucoside | mixture | | 10.50 | 0.0600 |
| Sodium Lauroyl | 137-16-6 | | 10.49 | 0.0600 |
| Sarcosinate | | | | |
| Butylene Glycol | 107-88-0 | | 3.18 | 0.0182 |
| PEG 14 M | 25322-68-3 | | 1.59 | 0.0091 |
| Fragrance | | | 0.80 | 0.0045 |
| Polyquaternium-10 | 53568-66-4 | | 0.80 | 0.0045 |
| Panthenol | 81-13-0 | | 0.64 | 0.0036 |
| Phenoxyethanol | 122-99-6 | | 0.48 | 0.0027 |
| Benzyl Alcohol | 100-51-6 | | 0.48 | 0.0027 |
| Methylparaben | 99-76-3 | | 0.40 | 0.0023 |
| Propylparaben | 94-13-3 | | 0.24 | 0.0014 |
| Disodium EDTA | 139-33-3 | | 0.16 | 0.0009 |
| Grape Seed Extract | mixture | | 0.02 | 0.0001 |
| Chamomile Extract | mixture | | 0.02 | 0.0001 |
| Aloe Vera Gel | | | 0.02 | 0.0001 |
| Vitamin E Acetate | 7695-91-2 | | 0.02 | 0.0001 |
| | TOTAL | 100.00 | 100.00 | 0.57126 |

### Example 2

Wet Non-Lathering Disposable Nonwoven Mitt

A mitt can be constructed by combining four nonwoven sheet members together. The first nonwoven sheet member can be comprised of hydroentangled polypropylene and rayon. The second nonwoven sheet member can be comprised of a needle punched rayon and polyester fibers and third nonwoven sheet member can be comprised of a batting material and the fourth nonwoven sheet members can be comprised of another bicomponent material. The bicomponent material is processed through a selective mechanical deformation process prior to combination of the four nonwoven sheet members. The four nonwoven sheet members are then bonded together using an ultrasonic bonder in a shape described in the Figures above and then rotary cut into individual units. After ultrasonic bonding but prior to die cutting into individual units the products are wetted with 4 grams of the following personal care composition.

This mitt can be associated with the following personal care composition:

| | | Percent Composition | |
|---|---|---|---|
| CTFA NAME | | As | Chem. |
| * | CAS number | Added | Content |
| Water | | 93.7579 | 93.76 |
| Niacinamide | | 2.0000 | 2.00 |
| Glycerin | | 1.0000 | 1.00 |
| Hexylene Glycol | | 1.0000 | 1.00 |
| Dimethicone and Dimethiconol | | 0.5000 | 0.50 |
| Dimethicone | | 0.5000 | 0.50 |
| DMDM Hydantoin and | | 0.6000 | 0.60 |
| Iodopropynyl But | | | |
| Polysorbate 20 | | 0.1500 | 0.15 |
| Acrylates/Vinyl Isodecanoate | | 0.1500 | 0.15 |
| Crosspolymer | | | |
| Disodium EDTA | | 0.1000 | 0.10 |
| Dexpanthenol | | 0.1000 | 0.10 |
| Tocopheryl Acetate | | 0.1000 | 0.10 |
| Potassium Hydroxide | | 0.0210 | 0.02 |
| Fragrance | | 0.0200 | 0.02 |
| Salicylic Acid | | 0.0001 | 0.00 |
| Ascorbic Acid | | 0.0010 | 0.00 |
| | TOTAL | 100.0000 | 100.00000 |

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A disposable nonwoven mitt adapted to fit around at least one finger on a user's hand, said mitt comprising:
a first and second nonwoven sheet members, said first and second nonwoven members being secured to each other along at least a portion of a periphery of said nonwoven sheet members to form an interior volume between said nonwoven sheet members and an opening to receive said finger(s) of said user's hand; and
wherein at least one of said nonwoven sheet members is adapted to extend below a web of said user's hand when said mitt is placed on said finger(s) of said user's hand to form a flap portion of said mitt; and
wherein said flap portion is optionally grasped by a user's thumb during use.

2. The disposable nonwoven mitt of claim 1, wherein said first and second nonwoven sheet members comprise fibers.

3. The disposable nonwoven mitt of claim 2, wherein said fibers are selected from the group consisting of natural fibers, synthetic fibers, and combinations thereof.

4. The disposable nonwoven mitt of claim 3, wherein said fibers are selected from the group consisting of monocomponent fibers, multicomponent fibers, multiconstituent fibers, capillary channel fibers, hollow fibers, shaped or lobed fibers, and combinations thereof.

5. The disposable nonwoven mitt of claim 3, wherein said combinations of said synthetic and natural fibers are selected from the group consisting of Tencel™ fibers, rayon fibers, and combinations thereof.

6. The disposable nonwoven mitt of claim 3, wherein said synthetic fibers are selected from the group consisting of nylon fibers, polyolefin fibers, polyester fibers, and combinations thereof.

7. The disposable nonwoven mitt of claim 3, wherein said natural fibers are selected from the group consisting of cellulosic, hemp, jute, cotton, and combinations thereof.

8. The disposable nonwoven mitt of claim 1, wherein said nonwoven sheet members are prepared by meltblowing, spunbonding, air-entanglement, hydroentanglement, thermal bonding, selective mechanical deformation and combination thereof.

9. The disposable nonwoven mitt of claim 1, further comprising a personal care composition associated with at least one of said first and second nonwoven sheet members.

10. The disposable nonwoven mitt of claim 9, wherein said personal care composition comprises skin treatment agents, skin conditioning agents, lathering cleansing agents, non-lathering cleansing agents, or mixtures thereof.

11. The disposable nonwoven mitt of claim 10, wherein said skin treatment agents are selected from the group consisting of vitamins, zeolites, peptides, sunscreen actives, terpene alcohols, desquamation actives, anti-acne actives, anti-wrinkle actives, anti-atrophy actives, anti-oxidants, flavanoids, anti-inflammatory agents, anti-cellulite agents, topical anesthetics, tanning actives, chelators, skin lightening agents, antimicrobial actives, anti-fungal actives, skin soothing actives, skin healing actives, skin moisturizing actives, cosmetic actives, and mixtures thereof.

12. The disposable nonwoven mitt of claim 10, wherein said skin conditioning agents are selected from the group consisting of petrolatum, fatty acids, esters of fatty acids, fatty alcohols, ethoxylated alcohols, polyol polyesters, glycerine, glycerin mono-esters, glycerin polyesters, epidermal and sebaceous hydrocarbons, lanolin, straight and branched hydrocarbons, silicone oil, silicone gum, vegetable oil, vegetable oil adduct, hydrogenated vegetable oils, nonionic polymers, natural waxes, petrolatum, petrolatum derivatives, synthetic waxes, polyolefinic glycols, polyolefinic monoester, polyolefinic polyesters, cholesterols, cholesterol esters, and mixtures thereof.

13. The disposable nonwoven mitt of claim 10, wherein said lathering cleansing agents comprise a surfactant, wherein said surfactant is selected from the group consisting of anionic surfactant selected from the group consisting of sarcosinates, sulfates, isethionates, phosphates, taurates, lactylates, glutamates, soaps, and mixtures thereof; nonionic surfactant selected from the group consisting of amine oxides, alkyl glucosides, alkyl polyglucosides, polyhydroxy fatty acid amides, alkoxylated fatty acid esters, sucrose esters, and mixtures thereof; amphoteric surfactant selected from the group consisting of betaines, sultaines, hydroxysultaines, alkyliminoacetates, iminodialkanoates, aminoalkanoates, and mixtures thereof.

14. The disposable nonwoven mitt of claim 10, wherein said non-lathering cleansing agents comprises a surfactant, wherein said surfactant is selected from the group consisting of polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, PEG-100 stearate, polyoxyethylene 20 sorbitan trioleate (Polysorbate 85), sorbitan monolaurate, polyoxyethylene 4 lauryl ether sodium stearate, polyglyceryl-4 isostearate, hexyl laurate, steareth-20, ceteareth-20, PPG-2 methyl glucose ether distearate, ceteth-10, diethanolamine cetyl phosphate, glyceryl stearate, PEG-100 stearate, and mixtures thereof.

15. The disposable nonwoven mitt of claim 1, wherein said exterior surface of said first nonwoven sheet member and exterior surface of said second nonwoven sheet member have different textures.

16. A disposable nonwoven mitt adapted to fit around at least one finger on a user's hand, said mitt comprising:
(a) a first and second nonwoven sheet members, said first and second nonwoven members being secured to each other along at least a portion of a periphery of said nonwoven sheet members to form an interior volume between said nonwoven sheet members and an opening to receive said finger(s) of said user's hand; and
(b) a personal care composition associated with at least one of said first and second nonwoven sheet members; and
wherein said personal care composition is substantially free of xanthan gum.

17. The disposable nonwoven mitt of claim 16, said personal care composition comprises skin treatment agents, skin conditioning agents, lathering cleansing agents, nonlathering cleansing agents, or mixtures thereof.

18. The disposable nonwoven mitt of claim 16, wherein said first and second nonwoven sheet members comprise fibers selected from the group consisting of natural fibers, synthetic fibers, and combinations thereof.

19. The disposable nonwoven mitt of claim 18, wherein said combinations of said synthetic and natural fibers are selected from the group consisting of Tencel™ fibers, rayon fibers, and combinations thereof.

20. The disposable nonwoven mitt of claim 18, wherein said synthetic fibers are selected from the group consisting of nylon fibers, polyolefin fibers, polyester fibers, and combinations thereof.

21. The disposable nonwoven mitt of claim 18, wherein said natural fibers are selected from the group consisting of cellulosic, hemp, jute, cotton, and combinations thereof.

22. The disposable nonwoven mitt of claim 16, wherein said exterior surface of said first nonwoven sheet member and said exterior surface of said second nonwoven sheet member have different textures.

23. The disposable nonwoven mitt of claim 16, wherein said nonwoven sheet members are prepared by meltblowing, spunbonding, air-entanglement, hydroentanglement, thermal bonding, selective mechanical deformation and combination thereof.

24. A disposable nonwoven mitt adapted to fit around at least one finger on a user's hand, said mitt comprising:
(a) first and second nonwoven sheet members; wherein at least one of said members is a multi-layer nonwoven sheet member; wherein said first and second nonwoven members are secured to each other along at least a portion of a periphery of said nonwoven sheet members to form an interior volume between said nonwoven sheet members and an opening to receive said finger(s) of said user's hand; and
(b) a personal care composition associated with at least one of said first and second nonwoven sheet members.

25. The disposable nonwoven mitt of claim 24, wherein said multi-layered nonwoven sheet member comprises at least one batting layer.

26. The disposable nonwoven mitt of claim 25, wherein said batting layer is selected from the group consisting of fibrous nonwovens, sponges, foams, reticulated foams, polymeric nets, scrims, vacuum-formed laminates, formed films, formed film composite material, and combinations thereof.

27. The disposable nonwoven mitt of claim 24, wherein said personal care composition comprises skin treatment agents, skin conditioning agents, lathering cleansing agents, non-lathering cleansing agents, or mixtures thereof.

28. The disposable nonwoven mitt of claim 24, wherein said first and second nonwoven sheet members comprises fibers selected from the group consisting of natural fibers, synthetic fibers, and combinations thereof.

29. The disposable nonwoven mitt of claim 28, wherein said combinations of said synthetic and natural fibers are selected from the group consisting of Tencel™ fibers, rayon fibers, and combinations thereof.

30. The disposable nonwoven cleansing mitt of claim 28, wherein said synthetic fibers are selected from the group consisting of nylon fibers, polyolefin fibers, polyester fibers, and combinations thereof.

31. The disposable nonwoven cleansing mitt of claim 28, wherein said natural fibers are selected from the group consisting of cellulosic, hemp, jute, cotton, and combinations thereof.

32. The disposable nonwoven mitt of claim 24, wherein at least one of said nonwoven sheet members is adapted to extend below a web of said user's hand when said mitt is placed on said finger(s) of said user's hand to form a flap portion of said mitt.

33. The disposable nonwoven mitt of claim 24, wherein said exterior surface of said first nonwoven sheet member and said exterior surface of said second nonwoven sheet member have different textures.

34. The disposable nonwoven mitt of claim 24, wherein said nonwoven sheet members are prepared by meltblowing, spunbonding, air-entanglement, hydroentanglement, thermal bonding, selective mechanical deformation and combination thereof.

35. A kit for a personal care mitt comprising:
(a) a personal care composition; and
(b) a disposable nonwoven mitt according to claim 1.

36. A method of cleansing or therapeutically treating skin, hair and other keratinous surfaces comprising the steps of:
a. associating the personal care composition with the nonwoven disposable mitt according to claim 1; and
b. contacting said skin, hair and other keratinous surfaces with a disposable nonwoven mitt; and
c. optionally rinsing said skin, hair and other keratinous surfaces with water.

37. A method of cleansing or therapeutically treating the skin, hair and other keratinous surfaces comprising the steps of:
(a) contacting said skin, hair and other keratinous surfaces with the nonwoven disposable mitt of claim 16; and
(b) optionally rinsing said skin, hair and other keratin-containing surfaces with water.

38. The method of claim 37, wherein said method further comprises the step of wetting said disposable nonwoven mitt with a fluid.
